(19) United States
(12) Patent Application Publication
(10) Pub. No.: US 2021/0247377 A1
(43) Pub. Date: Aug. 12, 2021

(54) BIOMEMORY FOR NANOPORE DEVICE AND METHODS OF MANUFACTURING SAME

(71) Applicant: PALOGEN, INC., Palo Alto, CA (US)

(72) Inventors: Kyung Joon Han, San Jose, CA (US); Jungkee Yoon, Santa Clara, CA (US)

(21) Appl. No.: 17/169,172

(22) Filed: Feb. 5, 2021

Related U.S. Application Data
(60) Provisional application No. 62/971,104, filed on Feb. 6, 2020.

(51) Int. Cl. G01N 33/487; H01L 29/423

(52) U.S. Cl. CPC G01N 33/48721; H01L 29/4234

(57) ABSTRACT

A nanopore device for characterizing biopolymer molecules includes first and second selecting layers having respective first and second pluralities of independently addressable inhibitory electrodes disposed along respective first and second axes of selection, where the second selecting layer is disposed adjacent the first selecting layer. The device also includes a third electrode layer having a third independently addressable electrode, where the third electrode layer is disposed adjacent the second selecting layer, such that the first and second selecting layers and the third electrode layer form a stack of layers along a Z axis and define a plurality of nanopore pillars. The first and second pluralities of inhibitory electrodes form an array, such that the first and second pluralities of inhibitory electrodes surround each of the plurality of nanopore pillars along the first and second axes of selection respectively.

### CROSS-REFERENCE TO RELATED APPLICATION

This application claims priority to U.S. Provisional Application No. 62/971,104, filed on Feb. 6, 2020 under attorney docket number PAL.30004.02 and, entitled “BIOMEMORY FOR NANOPORE DEVICE AND METHODS OF MANUFACTURING SAME,” the contents of which are hereby expressly and fully incorporated by reference in their entirety, as though set forth in full. This application includes subject matter similar to the subject matter described in co-owned U.S. Provisional Patent Application Ser. No. 62/566,313, filed on Sep. 29, 2017 under attorney docket number 165-101USIP and, entitled “MANUFACTURE OF THREE DIMENSIONAL NANOPORE DEVICE,” U.S. Provisional Patent Application Ser. No. 62/593,840, filed on Dec. 1, 2017 under attorney docket number BTL.30002.00 and, entitled “NANOPORE DEVICE AND METHOD OF MANUFACTURING SAME,” and U.S. Provisional Patent Application Ser. No. 62/612,534, filed on Dec. 31, 2017 under attorney docket number BTL.30003.00 and, entitled “NANOPORE DEVICE AND METHOD OF ELECTRICAL ARRAY ADDRESSING AND SENSING.” The contents of the above-mentioned applications are fully incorporated herein by reference as though set forth in full.

### FIELD OF THE INVENTION

The present invention relates generally to systems, devices, and processes for characterizing biopolymer molecules, and methods of manufacturing and using such systems and devices. In particular, the present invention relates to memory devices for nanopore sensors.

### BACKGROUND

Nucleic acid (e.g., DNA, RNA, etc.) sequencing is one of the most powerful methods to identify genetic variations at the molecular level. Many signatures of genetic diseases can be diagnosed by information collected through genome-wide single nucleotide polymorphisms (“SNPs”) analysis, gene fusion, genomic insertion and deletion, etc. These techniques and other molecular biology techniques require nucleic acid sequencing at some point. Current technologies to sequence nucleic acids at the single molecule level include a nanopore sequencing technology that has advantages over previous sequencing techniques because nanopore sequencing technology has the characteristics of a label-free and amplification-free technique that also has improved read lengths, and improved system throughput. Accordingly, nanopore sequencing technology has been incorporated into high-quality gene sequencing applications.

Early experimental systems for nanopore based DNA sequencing detected electrical behavior of ssDNA passing through an α-hemolysin (αHL) protein nanopore. Since then, nanopore based nucleic acid sequencing technology has been improved. For instance, solid-state nanopore based nucleic acid sequencing replaces biological/protein based nanopores with solid-state (e.g., semiconductor, metallic gates) nanopores, as described below.

A nanopore is a small hole (e.g., with a diameter of about 1 nm to about 100 nm) that can detect the flow of charged particles (e.g., ions, molecules, etc.) through the hole by the change in the ionic current and/or tunneling current. Because each nucleotide of a nucleic acid (e.g., adenine, cytosine, guanine, thymine in DNA, uracil in RNA) affects the electric current density across the nanopore in a specific manner as it physically passes through the nanopore, measuring changes in the current flowing through a nanopore during translocation results in data that can be used to directly sequence a nucleic acid molecule passing through the nanopore. As such, Nanopore technology is based on electrical sensing, which is capable of detecting nucleic acid molecules in concentrations and volumes much smaller than that required for other conventional sequencing methods. Advantages of nanopore based nucleic acid sequencing include long read length, plug and play capability, and scalability. However, current biological nanopore based nucleic acid sequencing techniques can require a fixed nanopore opening (e.g., with a diameter of about 2 nm), have poor sensitivity (i.e., unacceptable amount of false negatives), high cost that renders production worthy manufacturing a challenge, and strong temperature and concentration (e.g., pH) dependency.

With advancements in semiconductor manufacturing technologies, solid-state nanopores have become an inexpensive and superior alternative to biological nanopores partly due to the superior mechanical, chemical and thermal characteristics, and compatibility with semiconductor technology allowing the integration with other sensing circuitry and nanodevices. However, current nanopore DNA sequencing techniques (e.g., involving biological and/or solid-state nanopores) continue to suffer from various limitations, including low sensitivity and high manufacturing cost. FIG. 1 schematically depicts a state-of-art solid-state based 2-dimensional (“2D”) nanopore sequencing device 100. While, the device 100 is referred to as “two dimensional,” the device 100 has some thickness along the Z axis.

Many of the limitations of nanopore DNA sequencing techniques result from the intrinsic nature of nanopore devices and techniques that must overcome the fast translocation speed and small size (e.g., height of about 0.34 nm and diameter of about 1 nm) of a single nucleotide. Conventional electronic instrumentation (e.g., nanoelectrodes) cannot resolve and sense such fast moving and small nucleotides using conventional nanopore based DNA sequencing techniques. Also, high manufacturing cost prevents wider applications of nanopore based DNA sequencing.

Further, due to size limitations, external memory (e.g., SRAM or EEPROM) is used with nanopore devices (see FIG. 1) to store/cache information related to detected electrical changes. FIG. 1 depicts a typical solid-state based SONOS or MONOS (Si-Oxide-Nitride-Oxide-Si or Metal-Oxide-Nitride-Oxide-Si) technology with an external memory. The external memory introduces performance and cost limitations, especially in real time sequencing applications. Real time sequencing applications can require an entire genome of an organism to be sequenced rapidly (e.g., in one hour). While parallel processing may be capable of such rapid sequencing, external memory related performance limitations may limit the maximum sequencing speed. As the degree of parallel processing increases, external memory related performance limitations result in more significant reduction of sequencing rates.

In order to address the some of these drawbacks (sensitivity and some of the manufacturing cost) of current state-of-art nanopore technologies, multi-channel nanopore array which allows parallel processing of biomolecule sequencing may be used to achieve label-free, amplification-free, and rapid sequencing. Examples of such multi-channel nanopore arrays are described in U.S. Provisional Patent Application Ser. Nos. 62/566,313 and 62/593,840, the contents of which have been previously incorporated by reference. Since there is no known approach to electrically address such multi-channel nanopore arrays, in order to direct charged particles (e.g., biomolecules) to specific channels in such multi-channel nanopore arrays, some arrays are coupled to microfluidic channels outside the array. Other arrays operate using optical bead techniques by applying labels to charged particles before loading into the array sequencing to direct charged particles to specific channels in such nanopore arrays. Electrically addressing and sensing individual nanopore channels within multi-channel nanopore arrays can facilitate more efficient and effective use of multi-channel nanopore arrays to achieve low cost and high throughput sequencing of charged particles (e.g., biomolecules).

There is a need for on-board memory for nanopore based sequencing systems and devices that address the memory related shortcomings of currently-available sensing configurations, particularly for parallel processing nanopore array based sequencing systems and devices, which can perform sequencing operations at high speed for real time sequencing applications.

There are many efforts to use nanopore device in arrays to improve manufacturing throughput and lower the cost for nanopore devices (e.g., for sensors). Optical means such as Total Internal Reflection Fluorescence (TIRF) microscopy have been used to detect pore blockade in many nanopores in parallel by monitoring the fluorescence signal from proteins, DNA and many other applications. Nanopore sequencing using ionic current recording in planar bilayers, utilizing enzymes has been developed by Oxford Nanopore Technologies with 512 active channels per chip (MiNIon™) introduced in 2015. Based on typical nanopore sequencing speeds (about 28 ms per nucleotide), in order to sequence a total of 3×10⁹ bases (with 10× coverage) in 15 minutes requires about one million (10⁶) nanopores. However, current state of art nanopore arrays operate at less that the full throughput capacity from parallel processing because of external memory related performance limitations described above. There is no known method currently available to store/cache information related to detected electrical changes in nanopore array devices with sufficient speed to meet real time biomolecule sequencing requirements.

### SUMMARY

Embodiments described herein are directed to nanopore based sequencing systems and methods of sensing using same. In particular, the embodiments are directed to various types (2D or 3D) of nanopore based sequencing systems, methods of using nanopore array devices, and methods of sensing using same.

In one embodiment, a nanopore device for characterizing biopolymer molecules includes a first selecting layer having a first plurality of independently addressable inhibitory electrodes disposed along a first axis of selection. The device also includes a second selecting layer having a second plurality of independently addressable inhibitory electrodes disposed along a second axis of selection orthogonal to the first axis of selection, where the second selecting layer is disposed adjacent the first selecting layer. The device further includes a third electrode layer having a third independently addressable electrode, where the third electrode layer is disposed adjacent the second selecting layer, such that the first selecting layer, the second selecting layer, and the third electrode layer form a stack of layers along a Z axis and define a plurality of nanopore pillars. The first and second pluralities of inhibitory electrodes form an array, such that the first plurality of inhibitory electrodes surround each of the plurality of nanopore pillars along the first axis of selection, and the second plurality of inhibitory electrodes surround each of the plurality of nanopore pillars along the second axis of selection.

In one or more embodiments, the plurality of nanopore pillars is disposed in an array of nanopore pillars along a plane orthogonal to the Z axis. Each of the first plurality of inhibitory electrodes may be independently addressable to select a respective row of nanopore pillars from the array of nanopore pillars. Each of the second plurality of inhibitory electrodes may be independently addressable to select a respective column of nanopore pillars from the array of nanopore pillars. One of the first plurality of inhibitory electrodes and one of the second plurality of inhibitory electrodes may be independently addressable to select a nanopore pillar from the array of nanopore pillars.

In one or more embodiments, the first and second pluralities of inhibitory electrodes are cross-patterned electrodes. Each pair of the first plurality of inhibitory electrodes may be independently addressable to select a respective row of nanopore pillars from the array of nanopore pillars. Each pair of the second plurality of inhibitory electrodes may be independently addressable to select a respective column of nanopore pillars from the array of nanopore pillars. Respective pairs of the first and second pluralities of inhibitory electrodes may be independently addressable to select a nanopore pillar from the array of nanopore pillars.

In one or more embodiments, the first and second pluralities of inhibitory electrodes are configured to select a nanopore pillar from the array of nanopore pillars by applying a first inhibitory bias to all of the first plurality of inhibitory electrodes except a first inhibitory electrode corresponding to a selected row and applying a second inhibitory bias to all of the second plurality of inhibitory electrodes except a second inhibitory electrode corresponding to a selected column. The first and second inhibitory biases may generate respective first and second electric fields sufficient to suppress ionic translocation.

In one or more embodiments, the first and second electrodes are independently addressable to modify a translocation rate through the plurality of nanopore pillars. Sufficiently high positive gate voltage applied to the first and second inhibitory electrodes compared to the anode to cathode (i.e., top to bottom chamber) bias will inhibit the ionic current flow to a level such that enables a column (first electrode plane) and row (second electrode plane) array addressing scheme. The third electrode may also be independently addressable to modify its ionic charge state and thus change the surface charge of the selected (by the first and second electrodes) nanopore channel from the plurality of nanopore pillars and modify a translocation rate therethrough.

The third through Nₜₕ electrode may be independently addressable through nanoelectrode gate modulation. While applying the positive Vpp on the anode electrode of the electrolyte in the top chamber, applying a counter (positive) gate voltage to the third electrode will decrease the translation rate by decreasing the ionic current flow.

In one or more embodiments, the third through Nth electrode is independently addressable to modify a translocation rate through the plurality of nanopore pillars. The third electrode may be independently addressable to modify a surface charge of a wall of a nanopore pillar from the plurality of nanopore pillars to modify a translocation rate therethrough. The third electrode may be independently addressable through nanoelectrode gate modulation. Applying a positive gate voltage to the third electrode may increase the translation rate. Applying a negative gate voltage to the third electrode may decrease the translation rate.

In one or more embodiments, the third through Nₜₕ electrode is independently addressable to sense a change in an electrical characteristic related to the plurality of nanopore pillars. The third through Nₜₕ electrode may be independently addressable to detect the electrical characteristic using resistive pulse sensing, current-voltage sensing, Coulter counter technique, ionic blockade current technique, tunneling current technique, plasmonic sensing, or optical sensing.

The third through Nₜₕ electrode may be independently addressable to apply a voltage pulse in a transverse direction to the plurality of nanopore pillars. The third electrode may be independently addressable to sense a transconductance change resulting from the voltage pulse.

In one or more embodiments, the third electrode is independently addressable to record an electrical characteristic. The third electrode may be independently addressable to read the recorded electrical characteristic. The third electrode may be independently addressable to reset the third electrode for recording a second electrical characteristic.

In one or more embodiments, the device also includes a fourth electrode layer having a fourth independently addressable electrode. The fourth electrode layer may be disposed adjacent an opposite side of the third electrode layer from the second selecting layer, such that the first selecting layer, the second selecting layer, the third electrode layer, and the fourth electrode layer form an expanded stack of layers along the Z axis and define the plurality of nanopore pillars. The third electrode may be independently addressable to sense a time of flight measurement based on a time interval between signals sensed at the third and fourth electrode layers.

In one or more embodiments, each of the first and second pluralities of inhibitory electrodes and the third electrode are all nanoelectrodes. The nanopore device may form part of a solid-state, biological, or hybrid system. The nanopore device may form part of a 3D system. The nanopore device may form part of a 2D system.

In another embodiment, a method of manufacturing and using a nanofluidic NAND transistor sensor array scheme comprising a plurality of nanopore channel pillars, a plurality of respective fluidic channels, a plurality of gate electrodes, a top chamber, and a bottom chamber includes placing a sensor substrate in an electrolyte solution comprising biomolecules and DNA. The method also includes placing first and second electrodes in the electrolyte solution in the top and bottom chambers (Vpp and Vss of the NAND transistor). The method further includes forming the plurality of nanopore channel pillars in the sensor substrate. Moreover, the method includes placing the plurality of gate electrodes in respective walls of the plurality of nanopore channel pillars. In addition, the method includes placing a plurality of gate insulators between the plurality of vertical nanopore channel pillars and the plurality of gate electrodes to separate the plurality of vertical nanopore channel pillars from the plurality of gate electrodes. The method also includes applying an electrophoretic bias in the first and second electrodes in the electrolyte solution in the top and bottom chambers. The method further includes applying a bias in the plurality of gate electrodes in the respective walls of the plurality of nanopore channel pillars. Moreover, the method includes detecting a change in an electrode current in the electrolyte solution caused by a change in a gate voltage. In addition, the method includes storing the charge in the SiN interface of the dielectric between the gate electrode and channel by applying the sufficiently high positive voltage to the electrode. The method includes removing the charge from the SiN interface by applying the sufficiently high negative voltage to the gate electrode. The method also includes detecting a change in a surface charge in a plurality of nanopore channel electrodes in the plurality of respective fluidic channels.

In one or more embodiments, the plurality of nanopore channel pillars forms part of a 3D or 2D system.

In one or more embodiments, storing the change in the surface current includes applying a positive bias to a gate electrode. The change in the surface current may apply the positive bias to the gate electrode. The method may also include reading the stored change in the surface current from the gate electrode. The method may further include applying a negative bias to the gate electrode to eject the electrons from the second SiN interface.

In still another embodiment, a method of manufacturing and using a nanofluidic NAND transistor sensor array scheme comprising a plurality of nanopore channel pillars, a plurality of respective fluidic channels, a plurality of gate electrodes, a top chamber, and a bottom chamber includes placing a sensor substrate in an electrolyte solution comprising biomolecules and DNA. The method also includes placing first and second electrodes in the electrolyte solution in the top and bottom chambers (Vpp and Vss of the nanofluidic NAND transistor). The method further includes forming the plurality of nanopore channel pillars in the sensor substrate. Moreover, the method includes placing the plurality of gate electrodes in respective walls of the plurality of nanopore channel pillars. In addition, the method includes placing a plurality of gate insulators between the plurality of vertical nanopore channel pillars and the plurality of gate electrodes to separate the plurality of vertical nanopore channel pillars from the plurality of gate electrodes. The method also includes applying an electrophoretic bias in the first and second electrodes in the electrolyte solution in the top and bottom chambers. Moreover, the method includes detecting a change in an electrode current in the electrolyte solution caused by a change in a gate voltage. The method further includes applying a bias in the plurality of gate electrodes in the respective walls of the plurality of nanopore channel pillars for the nanofluidic NAND memory operation (so called Biomemory). The Biomemory allows storing the key biomolecule information during Nanopore operation. The method includes applying a bias to gate electrodes to program and erase the individual memory cell. In addition, the method includes electrically storing the change in the electrode current as electrons in a first SiN interface. The method also includes detecting a change in a surface charge in a plurality of nanopore channel electrodes in the plurality of respective fluidic channels. The method further includes storing the change in the surface current as electrons in a SiN interface by applying the positive gate voltage.

The aforementioned and other embodiments of the invention are described in the Detailed Description which follows.

### BRIEF DESCRIPTION OF THE DRAWINGS

The drawings described below are for illustration purposes only. The drawings are not intended to limit the scope of the present disclosure. The drawings illustrate the design and utility of various embodiments of the present disclosure. It should be noted that the figures are not drawn to scale and that elements of similar structures or functions are represented by like reference numerals throughout the figures. In order to better appreciate how to obtain the recited and other advantages and objects of various embodiments of the disclosure, a more detailed description of the present disclosure will be rendered by reference to specific embodiments thereof, which are illustrated in the accompanying drawings. Understanding that these drawings depict only typical embodiments of the disclosure and are not therefore to be considered limiting of its scope, the disclosure will be described and explained with additional specificity and detail through the use of the accompanying drawings.

FIG. 1 schematically illustrates a prior art solid-state 2D nanopore device;

FIGS. 2, 3 and 4 schematically illustrate memory devices/cells according to various embodiments.

FIGS. 5, 6, 7 and 8 schematically illustrate 3D nanopore devices including memory cells according to various embodiments.

FIG. 9 schematically illustrates a 3D nanopore device including memory cells according to one embodiment including some details of its operation.

FIG. 10 is a table summarizing the voltage operation of the nano-pore device for the programming and read depicted in FIG. 9.

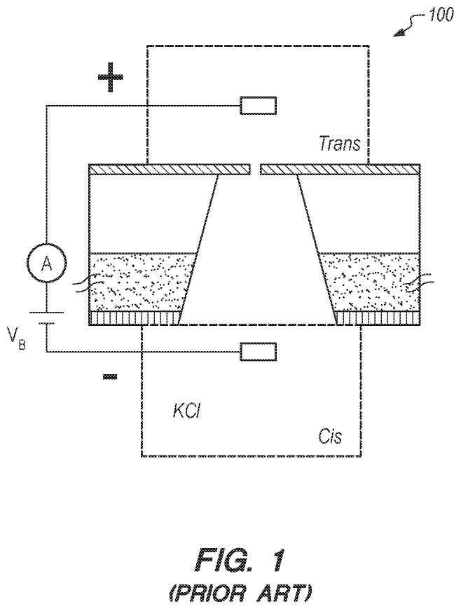

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

In order to address the above-described drawbacks (sensitivity and manufacturing cost) of current state-of-art nanopore technologies, multi-channel nanopore arrays that allow parallel processing of biomolecule sequencing may be used to achieve label-free, amplification-free, and rapid biomolecule sequencing. Examples of such multi-channel nanopore arrays are described in U.S. Provisional Patent Application Ser. Nos. 62/566,313 and 62/593,840, the contents of which have been previously incorporated by reference. Approaches to electrically address such multi-channel nanopore arrays, in order to direct charged particles (e.g., biomolecules) to specific channels in such multi-channel nanopore arrays, some arrays are coupled to microfluidic channels outside the array. Other arrays operate using optical bead techniques by applying labels to the charged particles before loading into the array for sequencing to direct charged particles to specific channels in such multi-channel nanopore arrays. Electrically addressing and sensing individual nanopore channels within multi-channel nanopore arrays, as described in U.S. Provisional Patent Application Ser. No. 62/612,534, the contents of which have been previously incorporated by reference, can facilitate more efficient and effective use of multi-channel nanopore arrays to achieve low cost and high throughput sequencing of charged particles (e.g., biomolecules).

In order to address the above-described memory related drawbacks (performance and manufacturing cost) of parallel processing nanopore technologies, electrode-based distributed memory systems are described herein to increase memory performance (e.g., capacity and speed) to facilitate rapid real time sequencing of biomolecules (e.g., an organisms entire genome in about an hour).

Memory devices and methods of efficiently and effectively storing/caching information related to detected electrical changes in nanopore array devices by applying a positive bias to an electrode to inject (program operation) electrons into a silicon nitride (“SiN”) interface and eject electrons (erase operation) by applying a negative voltages therein using same are described below. Such memory devices and methods can be used in various biomolecular arrays, including microarrays, CMOS arrays, and nanopore arrays (e.g., solid-state, biological, and hybrid nanopore arrays). Such memory devices and methods can also be used with various multi-channel nanopore arrays, including the 3D multi-channel nanopore arrays described above and planar multi-channel nanopore arrays.

Exemplary Memory Devices

As described above, current state-of-art nanopore devices are limited at least in terms of memory related performance limitations. The nanopore device embodiments described herein address, inter alia, these limitations of current nanopore devices.

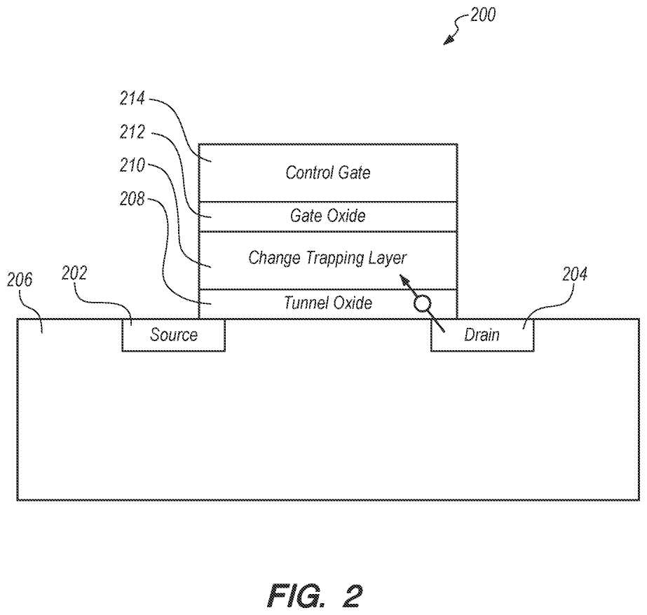

FIG. 2 schematically depicts a memory device (“cell”) 200 in a nanopore device according to one embodiment. The memory cell 200 includes a source 202 and a drain 204 embedded in substrate 206. The memory device 200 also includes various layers on top of the substrate 206, a tunnel oxide layer 208, a charge trapping layer (e.g., SiN) 210, a gate oxide layer 212, and a control gate layer 214. The charge trapping layer 210, which may include SiN, stores a change in a current (e.g., an electrode current) as electrons in the charge trapping layer 210. The electrons may be moved into the charge trapping layer 210 by applying a positive bias to the charge trapping layer 210 to pull the electrons into the charge trapping layer 210. Trapping the electrons places that particular memory cell 200 in an “off” state. However, other memory cells in an array will still be in an “on” state and ready to accept electrons. Memory cells such as the one described herein, can be used in nanopore devices such as nanopore arrays (2D or 3D). In nanopore arrays, each nanopore in the array can be associated with its own electrically isolated memory cell.

The electrons in the charge trapping layer 210 generate a charge that can be read. After the charge from the electrons is read, the memory cell 200 can be erased using a negative bias to eject the electrons from the memory cell 200. This resets the memory cell 200 to an “on” state, in which it is ready to record/store another change in a current. Various biases can be applied to these layers to perform the various memory cell functions (i.e., recording, reading, and erasing).

These layers may form a portion of an electrode in addition to the memory device 200. In such embodiments, when the electrode detects a current change, that current change may move electrons into the charge trapping layer 210 by causing a positive bias. Further, reading the charge from the electrons in the charge trapping layer 210 can eject the electrons and reset the memory cell 200 to the “on” state. The memory cell can be addressed in the manner described in U.S. Provisional Patent Application Ser. No. 62/612,534, the contents of which have been previously incorporated by reference.

The memory cell 200 is a NAND memory constructed in the nanofluidic channel, however, it is not restricted to use as a memory in nanopore devices (e.g., a nanopore channel). The memory cell 200 can be turned off when a programming bias is applied, and turned on/erased when an opposite polarity bias is applied. The memory cell 200 can also strongly inhibit nanofluidic channel leakage by applying a stronger programming bias (i.e., higher positive voltage on a gate electrode relative to a nanopore channel bias). This will inject more charges (e.g., electrons) into the gate electrode and turn off the memory cell 200. Any nanopore array (e.g., planar or 3D) can use the nanopore fluidic channel memory schemes described herein.

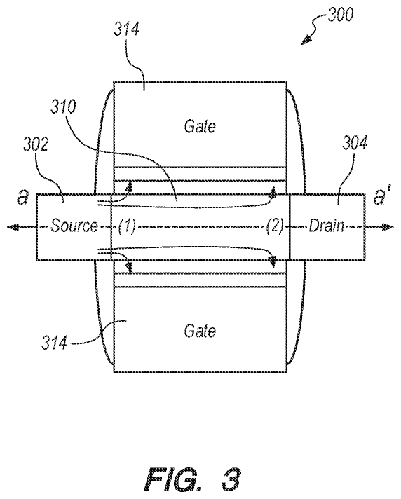

FIG. 3A schematically depicts a memory device (“cell”) 300 for use with a nanopore device according to another embodiment. In this embodiment, the charge trapping layer 310 is surrounded by two control gate layers 314 and directly electrically coupled to the source 302 and the drain 304.

FIG. 3B depicts the electrical addressing scheme for a memory device array 330 having a plurality of memory cells 300. The programming bias in Cell A is about 1 to 20V and the drain disturbance in Cell B is about 0V.

FIG. 3C depicts low and high drain disturbances using nickel silicide (“NiSi”) conductors and n⁺ conductors, respectively.

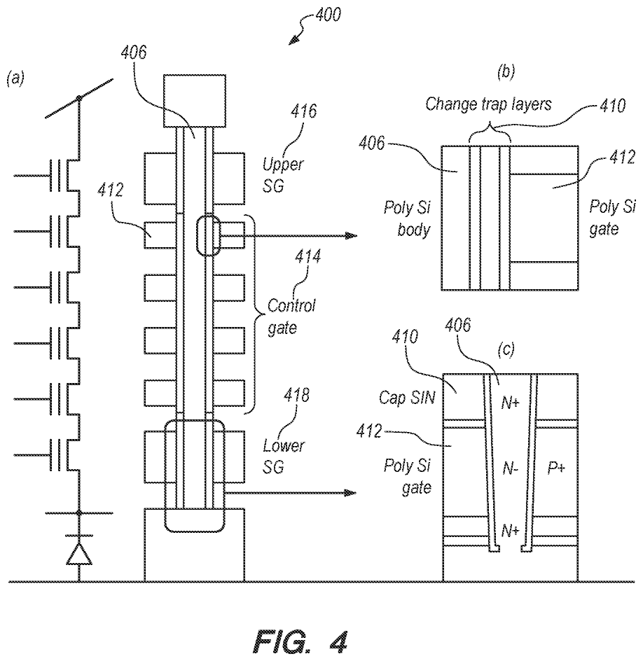

FIG. 4 schematically depicts a memory device (“cell”) 400 for use with a nanopore device according to another embodiment. In this embodiment, the memory cell 400 includes a plurality of elongated charge trapping layers 410. The charge trapping layers 410 are surrounded by a poly-silicon body 406 and a plurality of poly-silicon gates 412. FIG. 4 also depicts the electrical addressing scheme for the memory cell 400 including a plurality of poly-silicon gates 412. The memory cell 400 includes a control gate 414, and upper and lower SG gates 416, 418.

### Exemplary Nanopore Devices

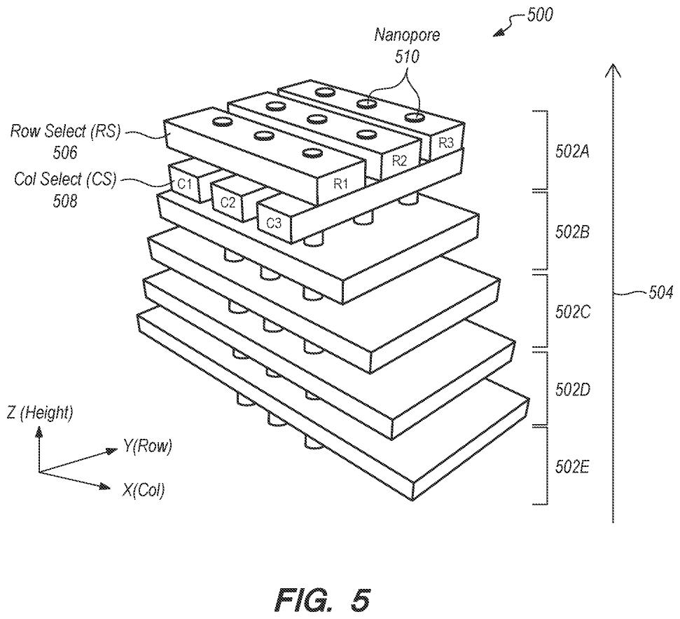

FIG. 5 schematically depicts a nanopore device 500 incorporating a plurality of memory cells (e.g., the memory cells 200, 300, 400 described above) with a three dimensional (“3D”) array architecture according to one embodiment. The device 500 includes a plurality of 2D arrays or layers 502A-502E stacked along a Z axis 504. While the 2D arrays 502A-502E are referred to as “two dimensional,” each of the 2D arrays 502A-502E has some thickness along the Z axis.

The top 2D array 502A includes first and second selecting (inhibitory electrode) layers 506, 508 configured to direct movement of charged particles (e.g., biopolymers) through the nanopores 510 (pillars) formed in the first and second selecting layers 506, 508. The first selecting layer 506 is configured to select from a plurality of rows (R1-R3) in the 2D array 502A. The second selecting layer 508 is configured to select from a plurality of columns (C1-C3) in the 2D array 502A. In one embodiment, the first and second selecting layers 506, 508 select from the rows and columns, respectively, by modifying a charge adjacent the selected row and column and/or adjacent to the non-selected rows and columns. The other 2D arrays 502B-502E include rate control/current sensing electrodes. Rate control/sensing electrodes may be made of highly conductive metals, such as Au—Cr, TiN, TaN, Pt, Cr, Graphene, Al—Cu, etc. The rate control/sensing electrodes may have a thickness of about 0.34 to about 1000 nm. Rate control/sensing electrodes may also be made in the biological layer in hybrid nanopores.

The other 2D arrays 502B-502E also include memory cells (e.g., the memory cells 200, 300, 400 described above) operatively coupled to respective sensing electrodes therein. The memory cells may also form part of respective sensing electrodes. Each sensing electrode may be operatively coupled to a nanopore 510 pillar, such that each nanopore 510 pillar may be operatively coupled to a particular memory cell.

Hybrid nanopores include a stable biological/biochemical component with solid-state components to form a semi-synthetic membrane porin to enhance stability of the nanopore. For instance, the biological component may be an αHL molecule. The αHL molecule may be inserted into a SiN based 3D nanopore. The αHL molecule may be induced to take on a structure to ensure alignment of the αHL molecule with the SiN based 3D nanopore by apply a bias to an electrode (e.g., in the top 2D array 502A).

The nanopore device 500 has a 3D vertical pillar stack array structure that provides a much larger surface area for charge detection than that of a conventional nanopore device having a planar structure. As a charged particle (e.g., biopolymer) passes through each 2D array 502A-502E in the device, its charge can be detected with a detector (e.g., electrode) in some of the 2D arrays 502B-502E. Therefore, the 3D array structure of the device 500 facilitates higher sensitivity, which can compensate for a low signal detector/electrode. The integration of memory cells into the 3D array structure minimizes any memory related performance limitations (e.g., with external memory device). Further, the highly integrated small form factor 3D structure provides a high density nanopore array while minimizing manufacturing cost.

In use, the nanopore device 500 is disposed between and separating top and bottom chambers (not shown) such that the top and bottom chambers are fluidly coupled by the nanopore pillars 510. The top and bottom chambers include an electrode (e.g., Ag/AgCl₂, etc.) and electrolyte solutions (KCl) containing the charged particles (e.g., DNA) to be detected. Different electrode and electrolyte solutions can be used for the detection of different charged particles.

Electrophoretic charged particle translocation can be driven by applying a bias to electrodes disposed in a top chamber (not shown) adjacent the top 2D array 502A of the nanopore device 500 and a bottom chamber (not shown) adjacent the bottom 2D array 502E of the nanopore device 500. In some embodiments, the nanopore device 500 is disposed in a between top and bottom chambers (not shown) such that the top and bottom chambers are fluidly and electrically coupled by the nanopore pillars 510 in the nanopore device 500. The top and bottom chambers may contain the electrolyte solution.

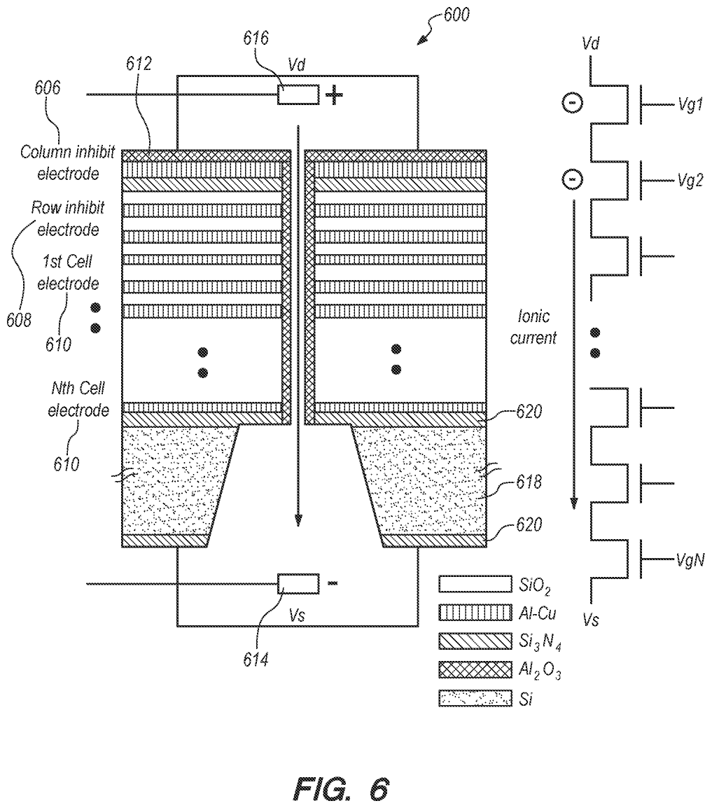

FIG. 6 schematically depicts a nanopore device 600 incorporating a plurality of memory cells (e.g., the memory cells 200, 300, 400 described above) according to one embodiment. The nanopore device 600 includes a column inhibitory electrode 606 (e.g., Al—Cu and Si₃N₄), a row electrode 608 (e.g., Al—Cu and SiO₂), and a plurality (1^{st} to N^{th}) of cell electrodes 610 (e.g., Al—Cu and SiO₂). The electrodes 606, 608, 610 of the nanopore device 600 are covered by an insulator dielectric film 612 (e.g., Al₂O₃). The nanopore device 600 also includes a source 614, a drain 616, and a substrate 618 (e.g., Si) between two dielectric layers 620 (e.g., Si₃N₄). FIG. 6 also depicts an exemplary electrical addressing scheme for the nanopore device 600 incorporating a plurality of memory cells according to one embodiment.

### Exemplary Multiple NP Memory Devices

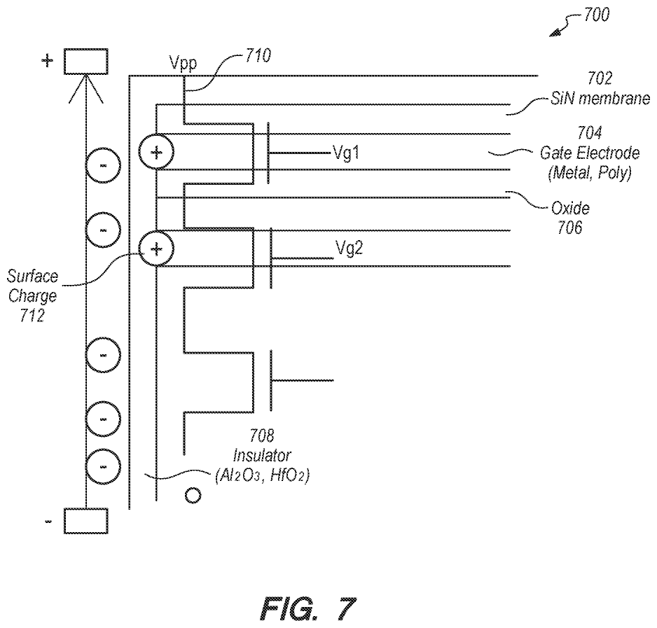

FIG. 7 schematically depicts a portion of a 3D nanopore sensor array 700 having a SiN membrane 702 on top of a transistor gate electrode (metal or polysilicon) 704 on top of an oxide 706. The gate electrode 704 can also be operatively coupled to/can form a memory cell (e.g., the memory cells 200, 300, 400 described above) for storing detected electrical characteristics (e.g., current, bias, etc.) This series 702, 704, 706 is repeated to form a stack of sensing electrodes/memory cells. The entire stack is covered with an insulator dielectric film (e.g., SiO₂, Al₂O₃, HfO, ZnO). The dielectric film's thickness is from about 2 nm to about 20 nm, and it can be a gate dielectric using SiO₂, Al₂O₃, or HfO. The thickness of the transistor gate electrode 704 is the channel length (gate film thickness in this case) of the transistor and it can be made with polysilicon or metals.

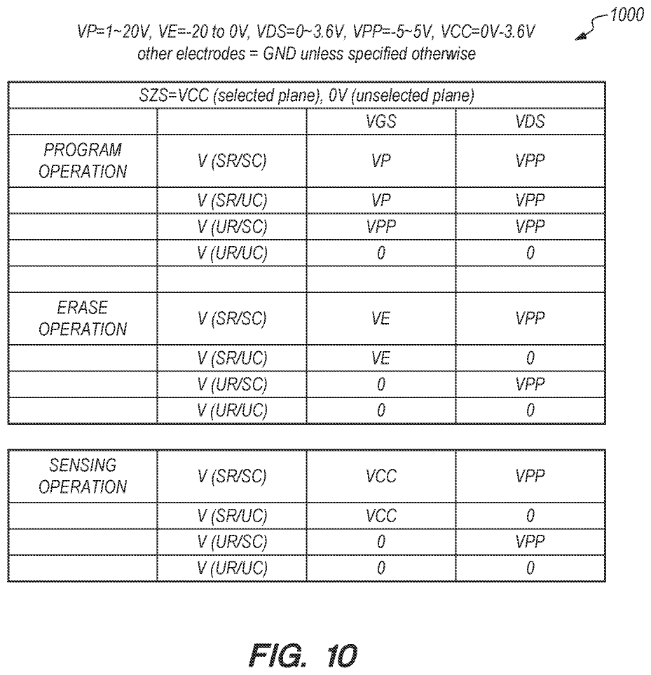

When a translocation rate control bias signal 710 for column and row voltages (e.g., Vpp, see “Normal Operation” in FIG. 10) is applied to the 3D nanopore sensor array 700, column and row Inhibitory voltage/bias pulses are followed by a verify (sensing) voltage/bias pulse (e.g., Vg1, Vg2), as described below. An exemplary signal 710 is depicted in FIG. 7 overlaid on top of the 3D nanopore sensor array 700. As described above with respect to “inhibitory operation” in FIG. 10, inhibitory biases are applied to deselect various column and row nanopore pillar channels, respectively. During sensing operation, both column and row inhibitory select electrodes are selected. The resulting surface charge 712 can be detected as a change in an electrical characteristic, such as current. This current can drive electrons into the gate electrode 704, which form memory cells for storing the detected current/surface charge 712.

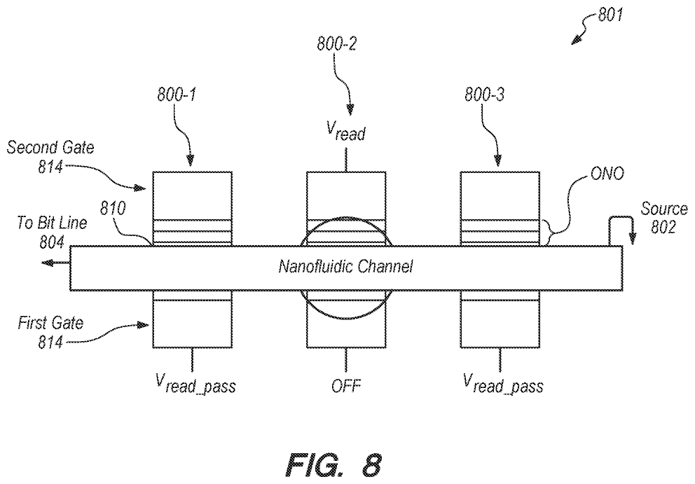

FIG. 8 schematically depicts a portion of a multiple memory cell device 801 according to another embodiment. The portion depicted in FIG. 8 includes three memory cells 800-1, 800-2, 800-3. Each memory cell 800-1, 800-2, 800-3 is similar to the memory cell 300 depicted in FIG. 3A. The charge trapping layer 810 is surrounded by first and second control gate layers 814 and directly electrically coupled to the source 802 and the drain 804 (“to bit line”). In the embodiment depicted I FIG. 8, the first and third memory cells 800-1, 800-3 are in the “on” state ready to read an electrical signal (e.g., current or bias). The second memory cell 800-2 is storing electrons corresponding to a read electrical signal and is in the “off” state.

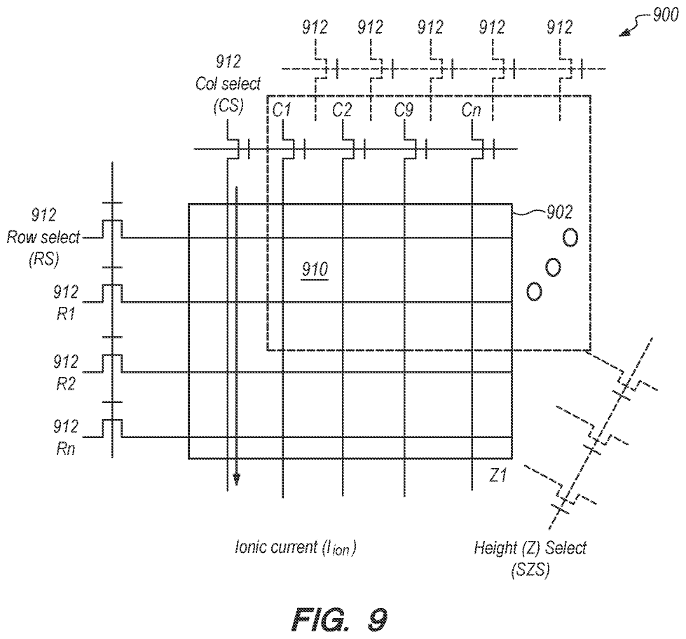

FIG. 9 schematically depicts a nanopore device 900 including memory cells according to another embodiment. FIG. 9 depicts the top 2D array 902 in a cross-sectional (x-z plane) view showing the 3D nanopore 910 and nanoelectrode schemes. Each nanopore 910 is surrounded by nanoelectrodes 912, allowing the nanopore 910 channel to operate under an electric bias field condition generated using the nanoelectrodes 912. Cross-patterned nanogap nanoelectrodes 912CS-912Cn, 912RS-912Rn are disposed in two layers on top of the nanopore device 900. These nanoelectrodes 912CS-912Cn, 912RS-912Rn are column and row inhibitory nanoelectrodes 912CS-912Cn, 912RS-912Rn for the nanopore array, respectively. The cross-patterned nanoelectrodes 912CS-912Cn, 912RS-912Rn as shown in the top 2D array 902 (x-y plane view) may be formed/patterned at the metal lithography steps. Nanoelectrodes 912 in the remaining 2D arrays in the 3D stack may be formed by plane depositing metals. These nanoelectrodes 912 can also be operatively coupled to/can form a memory cell (e.g., the memory cells 200, 300, 400 described above) for storing detected electrical characteristics (e.g., current, bias, etc.) The nanopore 910 hole pillars are surrounded by the metal nanoelectrodes 912CS-912Cn, 912RS-912Rn, and thus may operate under the full influence of the electrical bias applied to the multiple stacked nanoelectrodes 912.

By applying an inhibitory electrical bias (0V-VCC) to select nanogap nanoelectrodes 912CS-912Cn, 912RS-912Rn in the top 2D array 902, biomolecular translocation (e.g., electrophoretic) through one or more nanopores 902 in the top 2D nanopore array 902 can be inhibited to control nanopore array operation according to one embodiment. The electrical bias applied to the nanoelectrodes 912CS-912Cn, 912RS-912Rn can generate an electric field sufficient to suppress ionic translocation of charged particles (e.g., nucleic acids) from a top chamber (not shown) to a bottom chamber (not shown) in a direction orthogonal to the nanoelectrodes 912CS-912Cn, 912RS-912Rn. Nanoelectrode 912 mediated ionic translocation suppression can be substantially complete or the electrical bias can be modulated to only reduce the rate of ionic translocation. In one embodiment, after one or more nanopores 910 are selected (e.g., for DNA biomolecules translocation and sequencing), the electrical biases in a stack of 3D nanopore nanoelectrodes 912 can be modulated to control the biomolecular translocation speed. In one embodiment, the inhibitory electrical bias reduces/stops ionic current flow in the vertical direction to thereby select and/or deselect various columns and rows defined by the nanogap nanoelectrodes 912CS-912Cn, 912RS-912Rn.

At the same time, the nanoelectrodes 912 can detect current modulations resulting from passage of charged particles (e.g., DNA biomolecules) through the 3D vertical nanopore 910 pillars. In some embodiments, the nanoelectrodes 912 can detect current modulations using a variety of principles, including ion blockade, tunneling, capacitive sensing, piezoelectric, and microwave-sensing. As described above, the nanoelectrodes 912 either are operatively coupled to or form a memory cell (e.g., the memory cells 200, 300, 400 described above) for storing detected electrical characteristics (e.g., current, bias, etc.). Therefore, when the nanoelectrodes 912 can detect current modulations resulting from passage of charged particles (e.g., DNA biomolecules) through the 3D vertical nanopore 910 pillars, the detected current modulations may move electrons into the charge trapping layer of a memory cell to store an amount of charge corresponding to the current modulation. As described above, this amount of charge can be read, and then the memory cell can be erased using a negative bias.

Exemplary Nanopore Device Rate Control/Sensing Schemes

FIG. 10 is a table 1000 illustrating the voltage operation of a nanopore device including memory cells (e.g., the nanopore device 900 depicted in FIG. 9) according to various embodiments. As shown in FIG. 10, the nanopore memory device 900 can be operated in both program, erase and read modes by modulating the voltage/bias applied to various electrodes 912. VP (Program voltage) is from about 0V to about 20V; VE (Erase voltage) is about −20V to 0V, VD is from −5V to 5V, VCC is from about 0V to about 3.6V; and VSE is from about 0.1V to about 1.5V. All other electrodes are set to ground or floated unless other specified in the table in FIG. 10. The height select electrode (“SZS”; see FIG. 9) is set to VCC for the selected plane in the stack and to 0 form the unselected planes.

In program operation mode, the row and column voltages of the selected row (“SR”) and the selected column (“SC”) are set to VP and VD, respectively. The voltages of the unselected rows (“UR”) and unselected columns (“UC”) are set as shown in the table in FIG. 10.

In erase operation mode, the row and column voltages of the selected row (“SR”) and the selected column (“SC”) are set to VE and VD, respectively. The voltages of the unselected rows (“UR”) and unselected columns (“UC”) are set as shown in the table in FIG. 10.

In sensing operation mode, it follows the 3D Nanopore operation disclosed in the earlier applications. The row and column voltages of the selected row (“SR”) and the selected column (“SC”) are set to VCC and VSE, respectively. The voltages of the unselected rows (“UR”) and unselected columns (“UC”) are set as shown in the table in FIG. 10. The nanopore device 900 and the memory cells therein can be configured so that current modulations resulting from passage of charged particles (e.g., DNA biomolecules) through the 3D vertical nanopore 910 pillars move electrons into the charge trapping layer of a memory cell to store an amount of charge corresponding to the current modulation. As described above, this amount of charge can be read, and then the memory cell can be erased using a negative bias.

The memory cell devices described herein, when used with multi-channel nanopore array devices, allow real-time parallel processing of biomolecule interactions for rapid sequencing of biomolecules (e.g., whole genome sequencing in less than an hour). This facilitates label-free, amplification-free, rapid sequencing. In nanopore arrays, an on-board memory element facilitates signal and data processing in real time. Such memory elements may be used in various biomolecular array including but not limited to micro arrays, CMOS arrays, and nanopore arrays. The memory cells described herein perform the same function as external memory (e.g., SRAM or EEPROM) without the extra overhead in cost and performance.

As nanopore array size increases, a significant slowdown is expected due to serial operation of the array required by the lack of on-board memory elements in the biomolecular array. As such, the core advantages of nanopore array (e.g., parallel processing) cannot be fully realized to achieve low cost and high throughput nanopore biomolecule sequencing.

The memory cells disclosed herein allow effective storing of each cell information in a nanopore biosensor array, facilitating parallel addressing and sensing operations of the nanopore biomolecular array. Electrically injecting charges (either electrons or holes) through the nanoelectrodes embedded in the nanopore channel allows each nanoelectrode to function as separate memory cell. This solution can be used in any type of solid-state biomolecular array including but not limited to nanopore, CMOS, and microarray schemes.

The corresponding structures, materials, acts and equivalents of all means or step plus function elements in the claims below are intended to include any structures, materials, acts and equivalents for performing the function in combination with other claimed elements as specifically claimed. It is to be understood that while the invention has been described in conjunction with the above embodiments, the foregoing description and claims are not to limit the scope of the invention. Other aspects, advantages and modifications within the scope to the invention will be apparent to those skilled in the art to which the invention pertains.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. Other details of the present invention, these may be appreciated in connection with the above-referenced patents and publications as well as generally known or appreciated by those with skill in the art. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A nanopore device for characterizing biopolymer molecules, comprising:
a first selecting layer having a first plurality of independently addressable inhibitory electrodes disposed along a first axis of selection;
a second selecting layer having a second plurality of independently addressable inhibitory electrodes disposed along a second axis of selection orthogonal to the first axis of selection, wherein the second selecting layer is disposed adjacent the first selecting layer; and
a third electrode layer having a third independently addressable electrode, wherein the third electrode layer is disposed adjacent the second selecting layer, such that the first selecting layer, the second selecting layer, and the third electrode layer form a stack of layers along a Z axis and define a plurality of nanopore pillars,
wherein the first and second pluralities of inhibitory electrodes form an array, such that
the first plurality of inhibitory electrodes surround each of the plurality of nanopore pillars along the first axis of selection, and
the second plurality of inhibitory electrodes surround each of the plurality of nanopore pillars along the second axis of selection.

2. The device of claim 1, wherein the plurality of nanopore pillars is disposed in an array of nanopore pillars along a plane orthogonal to the Z axis.

3. The device of claim 2, wherein each of the first plurality of inhibitory electrodes is independently addressable to select a respective row of nanopore pillars from the array of nanopore pillars.

4. The device of claim 2, wherein each of the second plurality of inhibitory electrodes is independently addressable to select a respective column of nanopore pillars from the array of nanopore pillars.

5. The device of claim 2, wherein one of the first plurality of inhibitory electrodes and one of the second plurality of inhibitory electrodes are independently addressable to select a nanopore pillar from the array of nanopore pillars.

6. The device of claim 2, wherein the first and second pluralities of inhibitory electrodes are cross-patterned electrodes.

7. The device of claim 2, wherein each pair of the first plurality of inhibitory electrodes is independently addressable to select a respective row of nanopore pillars from the array of nanopore pillars.

8. The device of claim 2, wherein each pair of the second plurality of inhibitory electrodes is independently addressable to select a respective column of nanopore pillars from the array of nanopore pillars.

9. The device of claim 2, wherein respective pairs of the first and second pluralities of inhibitory electrodes are independently addressable to select a nanopore pillar from the array of nanopore pillars.

10. The device of claim 2, wherein the first and second pluralities of inhibitory electrodes are configured to select a nanopore pillar from the array of nanopore pillars by applying a first inhibitory bias to all of the first plurality of inhibitory electrodes except a first inhibitory electrode corresponding to a selected row and applying a second inhibitory bias to all of the second plurality of inhibitory electrodes except a second inhibitory electrode corresponding to a selected column, and
wherein the first and second inhibitory biases generate respective first and second electric fields sufficient to suppress ionic translocation.

11. (canceled)

12. The device of claim 1, wherein the third electrode is independently addressable to modify a translocation rate through the plurality of nanopore pillars,
wherein the third electrode is independently addressable to modify a surface charge of a wall of a nanopore pillar from the plurality of nanopore pillars to modify a translocation rate therethrough,
wherein the third electrode is independently addressable through nanoelectrode gate modulation,
wherein applying a positive gate voltage to the third electrode increases the translation rate, and
wherein applying a negative gate voltage to the third electrode decreases the translation rate.

13.-16. (canceled)

17. The device of claim 1, wherein the third electrode is independently addressable to sense a change in an electrical characteristic related to the plurality of nanopore pillars.

18. The device of claim 17, wherein the third electrode is independently addressable to detect the electrical characteristic using resistive pulse sensing, current-voltage sensing, Coulter counter technique, ionic blockade current technique, tunneling current technique, plasmonic sensing, or optical sensing.

19. The device of claim 17, wherein the third electrode is independently addressable to apply a voltage pulse in a transverse direction to the plurality of nanopore pillars, and
wherein the third electrode is independently addressable to sense a transconductance change resulting from the voltage pulse.

20. (canceled)

21. The device of claim 17, further comprising a fourth electrode layer having a fourth independently addressable electrode, wherein the fourth electrode layer is disposed adjacent an opposite side of the third electrode layer from the second selecting layer, such that the first selecting layer, the second selecting layer, the third electrode layer, and the fourth electrode layer form an expanded stack of layers along the Z axis and define the plurality of nanopore pillars
wherein the third electrode is independently addressable to sense a time of flight measurement based on a time interval between signals sensed at the third and fourth electrode layers.

22. The device of claim 17, wherein the third electrode is independently addressable to record an electrical characteristic,
wherein the third electrode is independently addressable to read the recorded electrical characteristic, and
wherein the third electrode is independently addressable to reset the third electrode for recording a second electrical characteristic.

23.-24. (canceled)

25. The device of claim 1, wherein each of the first and second pluralities of inhibitory electrodes and the third electrode are all nanoelectrodes.

26. The device of claim 1, wherein the nanopore device forms part of a solid-state, biological, or hybrid system.

27. The device of claim 1, wherein the nanopore device forms part of a 3D system.

28. The device of claim 1, wherein the nanopore device forms part of a 2D system.

29. The device of claim 1, wherein the first and second pluralities of inhibitory electrodes are formed using a lithography technique.

30. The device of claim 1, wherein the third electrode is formed using planar metal deposition.

31.-41. (canceled)
